(19) United States
(12) Patent Application Publication
(10) Pub. No.: US 2007/0117997 A1
(43) Pub. Date: May 24, 2007

(54) Process for the production of 4,4'-diphenylmethane diisocyanate

(75) Inventors: Berthold Keggenhoff, Krefeld (DE); Jeffrey Bolton, Dusseldorf (DE)

Correspondence Address: BAYER MATERIAL SCIENCE LLC, 100 BAYER ROAD, PITTSBURGH, PA, 15205, US

(73) Assignee: Bayer MaterialScience AG

(21) Appl. No.: 11/599,640

(22) Filed: Nov. 15, 2006

(30) Foreign Application Priority Data
Nov. 19, 2005 (DE) 102005055189.0

(51) Int. Cl. C07C 249/00

(52) U.S. Cl. 560/330

(57) ABSTRACT

The present invention relates to a process for the production of 4,4′-diphenylmethane diisocyanate (4,4′-MDI) by acid-catalyzed condensation of aniline with formaldehyde, reaction of the mixtures of di- and polyamines obtained with phosgene to form the corresponding mixture of MDI isomers and homologues (di- and polyisocyanates of the diphenylmethane series) and subsequent separation of the mixture by distillation to form 4,4′-MDI and polymeric MDI.

### FIELD OF THE INVENTION

The present invention relates to a process for the production of 4,4′-diphenylmethane diisocyanate (4,4′-MDI) by acid-catalyzed condensation of aniline with formaldehyde, reaction of the mixtures of di- and polyamines obtained with phosgene to form the corresponding mixture of MDI isomers and homologues (di- and polyisocyanates of the diphenylmethane series) and subsequent separation of the mixture by distillation to form 4,4′-MDI and polymeric MDI. During this process any secondary streams forming when the di- and polyisocyanate mixture is worked up to form 4,4′-MDI are preferably returned into the work-up of the polyisocyanate mixture and/or mixed in with the polymeric MDI, so that only technically pure 4,4′-MDI and commercial polymeric MDI are obtained as products of the process.

### BACKGROUND OF THE INVENTION

The industrial-scale production of 4,4′-MDI is conventionally carried out with the process steps of acid-catalyzed condensation of aniline with formaldehyde, reaction of the polyamine mixtures obtained with phosgene to form the mixture of MDI isomers and homologues (di- and polyisocyanates of the diphenylmethane series) and subsequent separation of the mixture by distillation to form technically pure 4,4′-MDI, polymeric MDI and other isomer mixtures. To this end, as described e.g. in EP-A-1475367, the mixture of MDI isomers and homologues (di- and polyisocyanates of the diphenylmethane series) is first produced. A partial distillate of isomeric diphenylmethane diisocyanates with 4,4′-MDI as the main component is then separated from this, the bottom product being used industrially as polymeric MDI. The partial distillate is then separated into pure 4,4′-MDI, a mixture of 4,4′-MDI with 2,4′-MDI and optionally 2,2′-MDI and other by-products. As described in WO-A-02/070581, 2,4′-MDI, for example, is obtained as the target product.

While there are markets in which 2,4′-MDI distillates are used for special applications, then, there are other markets and regions in which it is virtually impossible to sell these products since the special areas of application are lacking. For the production of 4,4′-MDI in particular, however, a production site in the immediate vicinity of the buyers' markets is highly advantageous because the product is solid at ambient temperature but can be stored in the liquid state for only a limited time owing to its tendency to form dimers, so that expensive transportation in the solid state with constant cooling is necessary to transport it over long distances. If there is then no demand for 2,4′-MDI in such a market, the problem arises of producing exclusively 4,4′-MDI and polymeric MDI as products of the process, in the ratio desired by the market and meeting the quality requirements specified by the market. At the same time, however, the other isomers necessarily forming during the condensation of aniline and formaldehyde, which are then reacted by phosgenation to form the corresponding isomers 2,2′- and 2,4′-MDI, must be put to a useful purpose.

### SUMMARY OF THE INVENTION

The present invention therefore provides a simple process for the production of 4,4′-MDI by acid-catalyzed condensation of aniline with formaldehyde, reaction of the polyamine mixtures obtained with phosgene to form the mixture of MDI isomers and homologues and subsequent separation of the mixture by distillation to form 4,4′-MDI and polymeric MDI, in which technically pure 4,4′-MDI and polymeric MDI are obtained as the only products of the process, in a technically conventional form and in a ratio that can be adjusted within a broad range.

These and other advantages and benefits of the present invention will be apparent from the Detailed Description of the Invention herein below.

### DETAILED DESCRIPTION OF THE INVENTION

The present invention will now be described for purposes of illustration and not limitation. Except in the operating examples, or where otherwise indicated, all numbers expressing quantities, percentages and so forth in the specification are to be understood as being modified in all instances by the term “about.”

The present invention provides a process for the production of 4,4′-diphenylmethane diisocyanate involving
a) reacting aniline and formaldehyde in a molar ratio of 1.7 to 4:1 in the presence of an acid catalyst to form di- and polyamines of the diphenylmethane series,
b) reacting the di- and polyamines with phosgene to form the corresponding di- and polyisocyanates of the diphenylmethane series and optionally separating by distillation, which results in a mixture of di- and polyisocyanates containing 44-80 wt. %, preferably 50-75 wt. %, particularly preferably 55-70 wt. % of 4,4′-diphenylmethane diisocyanate together with 1 to 12 wt. %, preferably 2-10 wt. %, particularly preferably 4-9 wt. % of 2,4′- and/or 2,2′-diphenylmethane diisocyanate in total, and 10 to 55 wt. %, preferably 20 to 48 wt. %, particularly preferably 26-40 wt. % of tri- and polyfunctional polyisocyanates, based on the weight of the mixture of di- and polyisocyanates, and
c) separating the mixture of di- and polyisocyanates by distillation and/or crystallization into precisely two fractions, the first fraction containing 5-40 wt. %, preferably 10-30 wt. % of the quantity of the mixture of di- and polyisocyanates and the second fraction containing 60-95 wt. %, preferably 70-90 wt. % of the quantity of the mixture of di- and polyisocyanates, and the first fraction containing at least 97 wt. % of 4,4′-diphenylmethane diisocyanate together with no more than 3 wt. % of 2,4′-diphenylmethane diisocyanate, preferably at least 98 wt. % of 4,4′-diphenylmethane diisocyanate together with no more than 2 wt. % of 2,4′-diphenylmethane diisocyanate and no more than 0.1 wt. % of 2,2′-diphenylmethane diisocyanate, based on the weight of the first fraction, and the second fraction containing 30-60 wt. % of 4,4′-diphenylmethane diisocyanate, 1-12 wt. % of 2,4′-diphenylmethane diisocyanate and no more than 2 wt. % of 2,2′-diphenylmethane diisocyanate together with 35-65 wt. % of tri- and polyfunctional polyisocyanates, preferably 35-55 wt. % of 4,4′-diphenylmethane diisocyanate, 2-10 wt. % of 2,4′-diphenylmethane diisocyanate and no more than 1 wt. % of 2,2′-diphenylmethane diisocyanate, together with 40-60 wt. % of tri- and polyfunctional polyisocyanates, based on the weight of the second fraction. The viscosity of this second fraction, measured at 25° C., is preferably 40 to 2,000 mPas, particularly preferably 100 to 800 mPas, measured in accordance with DIN 53015/ISO 12058.

In a preferred embodiment of the process, any secondary streams forming in step c) from the distillation and/or crystallization are added to the second fraction or recycled into the workup process of the mixture of di- and polyisocyanates in such a way that they finally remain in this second fraction.

The industrial production of di- and polyamines of the diphenylmethane series (MDA) in step a) generally takes place in two steps, with an acid-catalyzed condensation of aniline with formaldehyde initially taking place in the first step to produce the corresponding MDA mixture. A strong mineral acid, such as aqueous hydrochloric acid, is conventionally employed as the acid catalyst. A number of processes for the production of MDA by acid-catalyzed aniline-formaldehyde condensation are known (e.g. WO-A-99/40059, WO-A-99/54289).

It is possible to control the proportions of 4,4′-diphenylmethane diisocyanate and its isomers and homologues through the selection of the quantitative ratios of aniline, formaldehyde and mineral acid and of the temperature and residence time conditions. It has been shown that, by varying the molar ratio of aniline and formalin in the range of 1.7:1 to 4:1 when using 0.08 to 0.3 moles of mineral acid per mole of aniline, the ratio of the end products 4,4′-MDI to polymeric MDI can be varied in the range of 5:95 parts by weight to 40:60 parts by weight without causing any noticeable change in the application-specific properties of the polymeric MDI.

It is possible to carry out the condensation on an industrial scale both continuously and batchwise.

In a second step, the reaction solution obtained is worked up to form MDA by distillation of water and aniline, optionally after neutralization and phase separation.

In step b) the di- and polyamines of the diphenylmethane series are converted to the corresponding di- and polyisocyanates of the diphenylmethane series by reaction with phosgene. This reaction usually takes place in an inert solvent such as chlorobenzene, dichlorobenzene or toluene, with solutions of amine and phosgene being mixed and then fully reacted by heating. The hydrogen chloride also formed is usually separated off together with excess phosgene, and the solvent used is separated off, generally stepwise. After partial, or preferably complete, separation of the solvent, the di- and polyisocyanates of the diphenylmethane series are obtained as the bottom product. Processes for the production of di- and polyisocyanates of the diphenylmethane series by reacting di- and polyamines of the diphenylmethane series with phosgene are also known in the art (e.g. WO-A-99/54289). In step b), optionally after working up by distillation, a mixture of di- and polyisocyanates containing 44-80wt. %, preferably 50-75 wt. %, particularly preferably 55-70 wt. %, of 4,4′-diphenylmethane diisocyanate, together with 1 to 12 wt. %, preferably 2-10 wt. %, particularly preferably 4-9 wt. %, of 2,4′- and/or 2,2′-diphenylmethane diisocyanate in total and 10 to 55 wt. %, preferably 20 to 48 wt. %, particularly preferably 26-40 wt. % of tri- and polyfunctional polyisocyanates, based on the weight of the mixture of di- and polyisocyanates, is thus obtained.

In step c), two fractions are separated from the mixtures of di- and polyisocyanates containing 44-80 wt. % of diphenylmethane diisocyanate together with 1 to 12 wt. % of 2,4′- and/or 2,2′-diphenylmethane diisocyanate in total and 10 to 55 wt. % of tri- and polyfunctional polyisocyanates, based on the weight of the mixture of di- and polyisocyanates, by distillation and/or crystallisation (e.g. according to DE-A-1938384, DE-A-2631168, EP-A-79516, EP-A-1475367), the first fraction containing at least 97 wt. % of 4,4′-diphenylmethane diisocyanate together with no more than 3 wt. % of 2,4′-diphenylmethane diisocyanate, preferably at least 98 wt. % of 4,4′-diphenylmethane diisocyanate together with no more than 2 wt. % of 2,4′-diphenylmethane diisocyanate and no more than 0.1 wt. % of 2,2′-diphenylmethane diisocyanate, based on the weight of the first fraction, and the second fraction containing 30-60 wt. % of 4,4′-diphenylmethane diisocyanate, 1-12 wt. % of 2,4′-diphenylmethane diisocyanate and no more than 2 wt. % of 2,2′-diphenylmethane diisocyanate together with 35-65 wt. % of tri- and polyfunctional polyisocyanates, preferably 35-55 wt. % of 4,4′-diphenylmethane diisocyanate, 2-10 wt. % of 2,4′-diphenylmethane diisocyanate and no more than 1 wt. % of 2,2′-diphenylmethane diisocyanate together with 40-60 wt. % of tri- and polyfunctional polyisocyanates, based on the weight of the second fraction. The viscosity of this second fraction, measured at 25° C., is preferably-40 to 2,000 mPas, particularly preferably 100 to 800 mPas, measured in accordance with DIN 53015/ISO 12058.

This distillation and/or crystallization can contain several distillation and/or crystallization steps. During this distillation and/or crystallization, two product streams are removed exclusively and, in particular, the removal of substantially monomeric streams (i.e. containing diisocyanate) containing relatively large quantities of 2,4′-MDI is avoided, as these substantially monomeric streams (i.e. containing diisocyanate) containing relatively large quantities of 2,4′-MDI are difficult to market in some regions.

The process is preferably carried out as follows: the first process step preferably involves a partial separation of the diisocyanates (monomeric MDI) by distillation and/or crystallization, which results in a bottom product or bottom stream containing di- and polyisocyanates and a fraction containing at least 95 wt. % of diisocyanates, based on the weight of the fraction (polymer/monomer separation). The separated fraction containing at least 95 wt. % of diisocyanates (monomeric MDI) may already meet the quality requirements for technically pure 4,4′-MDI, i.e. at least 97 wt. % of 4,4′-diphenylmethane diisocyanate and no more than 3 wt. % of 2,4′-diphenylmethane diisocyanate and no more than 0.5 wt. % of other constituents, based on the weight of the fraction, and in this case it is removed directly as the first fraction. However, if the separated fraction containing at least 95 wt. % of diisocyanates (monomeric MDI) fails to meet the quality requirements for technically pure 4,4′-MDI, the fraction containing at least 95 wt. % of diisocyanates (monomeric MDI) is preferably freed at least partly from the 2,2′- and 2,4′-isomers and other impurities in further distillation and/or crystallization steps, wherein secondary streams containing 2,2′-MDI, 2,4′-MDI and/or other impurities are formed. However, as a product of the process, technically pure 4,4′-MDI containing at least 97 wt. % of 4,4′-diphenylmethane diisocyanate and no more than 3 wt. % of 2,4′-diphenylmethane diisocyanate, based on the weight of the fraction, is obtained as the first fraction in any case.

The bottom product or bottom stream containing di- and polyisocyanates is combined with the secondary streams that may have formed during the distillation and/or crystallization and processed into conventional commercial polymeric MDI in different viscosities in the range of 40 to 2,000 mPas, usually 100 to 800 mPas, measured in accordance with DIN 53015/ISO 12058 at 25° C. in each case. Depending on the quality of the individual streams, this can take place in the simplest case by simple mixing, the second fraction being obtained in this way. However, it may also be advantageous to heat the mixture again and to subject it to another distillation, preferably a flash distillation, or to return the secondary streams to another point in the work-up process of the mixture of di- and polyisocyanates, the second fraction containing 30-60 wt. % of 4,4′-diphenylmethane diisocyanate, 1-12 wt. % of 2,4′-diphenylmethane diisocyanate and no more than 2 wt. % of 2,2′-diphenylmethane diisocyanate together with 35-65 wt. % of tri- and polyfunctional polyisocyanates, preferably 35-55 wt. % of 4,4′-diphenylmethane diisocyanate, 2-10 wt. % of 2,4′-diphenylmethane diisocyanate and no more than 1 wt. % of 2,2′-diphenylmethane diisocyanate together with 40-60 wt. % of tri- and polyfunctional polyisocyanates, based on the weight of the second fraction, being obtained as the bottom product in all cases.

The distillate can be mixed in again with the fraction containing at least 95 wt. % of diisocyanates removed in step c), preferably in the polymer/monomer separation. Finally, it is also possible for all the bottom and distillate streams, except the first fraction, to be mixed in again with the mixture of di- and polyisocyanates of the diphenylmethane series obtained in step b), and then to obtain as the second fraction in step c) conventional commercial polymeric MDI as the bottom product in the first process step, preferably with the desired viscosity in the range of 40 to 2,000 mPas, usually 100 to 800 mPas. In principle, of course, it is also possible to add individual secondary streams from the distillation and/or crystallization to the fraction containing at least 95 wt. % of diisocyanates or to the first fraction, provided that it is ensured that the first fraction meets the quality requirements for technically pure 4,4′-MDI, i.e. at least 97 wt. % of 4,4′-diphenylmethane diisocyanate and no more than 3 wt. % of 2,4′-diphenylmethane diisocyanate, based on the weight of the fraction, and that a total of precisely two fractions, i.e. the first fraction and the second fraction, are obtained and removed.

The process according to the invention is made possible by the knowledge that the ratio of the proportions of the various diisocyanate isomers (2,2′-, 2,4′- and 4,4′-MDI) to one another and to the quantity of the tri- and polyfunctional polyisocyanates in the second fraction (polymeric MDI) can be varied within certain limits without changing the application-specific properties of the polymeric MDI to any great extent. This is the reason for the specification given for the second fraction of 30-60 wt. % of 4,4′-diphenylmethane diisocyanate, 1-12 wt. % of 2,4′-diphenylmethane diisocyanate and no more than 2 wt. % of 2,2′-diphenylmethane diisocyanate together with 35-65 wt. % of tri- and polyfunctional polyisocyanates, preferably 35-55 wt. % of 4,4′-diphenylmethane diisocyanate, 2-10 wt. % of 2,4′-diphenylmethane diisocyanate and no more than 1 wt. % of 2,2′-diphenylmethane diisocyanate together with 40-60 wt. % of tri- and polyfunctional polyisocyanates, based on the weight of the second fraction.

This also makes it possible to recycle the secondary streams forming during the distillation and/or crystallization and to refrain from discharging substantially monomeric streams (i.e. containing diisocyanate) containing relatively large quantities of 2,4′-MDI. Instead, the 2,4′-MDI predominantly acts as a constituent of the second fraction and is discharged with the second fraction, and so the 2,4′-MDI does not have to be isolated by technically complex means, transported and/or put to another use.

The following examples are intended to explain the invention in more detail but without limiting the scope thereof.

### EXAMPLES

### Example 1

a) Production of a Mixture of Di- and Polyamines:

2,600 g of aniline was mixed intensively with 1,000 g of formalin (30 wt. % aqueous solution of formaldehyde, based on the weight of the solution) in a stirred vessel at 25° C., with stirring, during which the mixture heated up to 60° C. The stirrer was turned off and the aqueous phase settling out at the top was separated off. 680 g of 30 wt. % aqueous hydrochloric acid was mixed in, while stirring again and cooling, maintaining a temperature of 45° C. After stirring at this temperature for a further 15 min, the cooling was replaced by heating and the mixture was uniformly heated to 140° C. in the course of 120 min under 5 bar pressure, and was maintained at this temperature for 15 min.

The mixture was cooled to 100° C., depressurized to normal pressure and neutralized by adding 540 g of 50 wt. % aqueous sodium hydroxide solution, while stirring. After turning off the stirrer, the phases were left to settle and the lower aqueous phase was removed by suction. Excess aniline was distilled off with residual water, initially under normal pressure, and the aniline residues were removed by starting to distil the polyamine mixture obtained at 100 mbar and 250° C.

1,900 g of a mixture of di- and polyamines of the following composition were obtained:

4,4-MDA: 60.1 wt. %,

2,4′-MDA: 6.0 wt. %,

2,2′-MDA: 0.2 wt. %,

higher molecular weight polyamines: 33.7 wt. %, based on the weight of the mixture.

b) Production of a Mixture of Di- and Polyisocyanates

The 1,900 g of the mixture of di- and polyamines obtained in Example 1 a) were dissolved in 5,700 g of chlorobenzene in another stirred reactor. In a second vessel, a 33 wt. % (based on the weight of the solution) phosgene solution was prepared by dissolving 3,800 g of phosgene in 7,600 g of chlorobenzene, while cooling to 0° C., and the amine and phosgene solutions were mixed while stirring intensively. The resulting suspension of solids was heated slowly with the formation of hydrogen chloride gas, which was withdrawn in a suitable manner. A homogeneous solution of polyisocyanate was formed during this process. The solvent was separated off by distillation, which resulted in 2,370 g of a mixture of di- and polyisocyanates of the following composition being obtained:

4,4′-MDI: 59.3 wt. %,

2,4′-MDI: 5.5 wt. %,

2,2′-MDI: 0.2 wt. %,

higher molecular weight polyisocyanates: 35 wt. %, based on the weight of the mixture.

c) Production of the First Fraction (Technically Pure 4,4′-MDI):

The mixture of di- and polyisocyanates obtained in Example 1 b) was distilled in a batch distillation under a pressure of 10 mbar and at a bottom temperature of 215° C. until 950 g of distillate (fraction containing at least 95 wt. % of diisocyanates) were obtained, at which point 1,420 g of a bottom product with a viscosity of 400 mPas remained.

The distillate (fraction containing at least 95 wt. % of diisocyanates) was worked up further in a divided wall column corresponding to EP-A-1475367. In this process, 59 g/h of the distillate from the batch distillation were fed to the divided wall column in the area of the wall. A bottom stream of 51 g/h with a 4,4′-MDI content of 98 wt. % and a 2,4′-MDI content of 2 wt. % was removed from the divided wall column and discharged as the first fraction. In addition, an overhead stream of 0.7 g/h with a composition of 25 wt. % 2,2′-MDI, 73 wt. % 2,4′-MDI and 2 wt. % 4,4′-MDI was removed together with a side stream of 6.1 g/h with a composition of 42.6 wt. % 2,4′-MDI and 57.4 wt. % 4,4′-MDI.

Gauze packings with a specific surface area of 500 m²/m³ were used as mass transfer elements in the partition column. The rectifying zone and the stripping zone possessed eight (8) separation stages each, the pre-fractionation zone and the main fractionation zone 12 separation stages each at the top and bottom, i.e. above and below the input of the feed stream into the pre-fractionation zone or the side stream removal from the main fractionation zone. The top pressure was 6 mbar. The return at the distillate discharge was 90:1 and at the side stream discharge 2.6:1.

d) Production of the Second Fraction (Polymeric MDI):

The secondary streams obtained in Example 1 c), i.e. the overhead stream (11.5 g) and the side stream (98.5 g) were mixed with the bottom stream (1420 g) also obtained in Example 1 c), as a result of which 1,530 g of polymeric MDI were obtained as the second fraction.

In total, therefore, 822 g of technically pure 4,4′-MDI were obtained as the first fraction and 1,530 g of polymeric MDI with a viscosity of 200 mPas as the second fraction.

### Example 2

a) Production of a Mixture of Di- and Polyamines:

1,800 g of aniline were mixed intensively with 1,000 g of formalin (30 wt. % aqueous solution of formaldehyde, based on the weight of the solution) in a stirred vessel at 30° C., with stirring, during which the mixture heated up to 80° C. The stirrer was turned off and the aqueous phase settling out at the top was separated off. 230 g of 30 wt. % aqueous hydrochloric acid were mixed in, while stirring again and cooling, maintaining a temperature of 45° C. After stirring at this temperature for a further 15 min, the cooling was replaced by heating and the mixture was uniformly heated to 140° C. in the course of 150 min under 5 bar pressure, and was maintained at this temperature for 20 min.

The mixture was cooled to 100° C., depressurized to normal pressure and neutralized by adding 180 g of 50 wt. % aqueous sodium hydroxide solution, while stirring. After turning off the stirrer, the phases were left to settle and the lower aqueous phase was withdrawn by suction. Excess aniline was distilled off with residual water, initially under normal pressure, and the aniline residues were removed by starting to distil the polyamine mixture obtained at 100 mbar and 250° C.

1880 g of a mixture of di- and polyamines of the following composition were obtained:

4,4′-MDA: 44.5 wt. %,

2,4′-MDA: 7.3 wt. %,

2,2′-MDA: 0.5 wt. %,

higher molecular weight polyamines: 47.7 wt. %, based on the weight of the mixture.

b) Production of the Polyisocyanate Mixture:

The mixture of di- and polyamines obtained in Example 2 a) was reacted with phosgene in chlorobenzene in the same way as described in Example 1, with the same molar ratio of phosgene to amino groups as in Example 1, to form the polyisocyanate mixture.

2,330 g of a polyisocyanate mixture of the following composition were obtained:

4,4′-MDI: 44.1 wt. %,

2,4′-MDI: 7.2 wt. %,

2,2′-MDI: 0.5 wt. %,

higher molecular weight polyisocyanates: 48.2 wt. %, based on the weight of the mixture.

c) Production of the First Fraction (Technically Pure 4,4′-MDI):

The mixture of di- and polyisocyanates obtained in Example 2 b) was distilled in a batch distillation under a pressure of 10 mbar and at a bottom temperature of 215° C. until 280 g of distillate (fraction containing at least 95 wt. % of diisocyanates) were obtained, at which point 2,050 g of bottom product remained.

The distillate (fraction containing at least 95 wt. % of diisocyanates) was worked up further in a divided wall column corresponding to EP-A-1475367 as in Example 1. In this process, 59 g/h of the distillate from the batch distillation were fed to the divided wall column in the area of the wall. A bottom stream of 46.9 g/h with a 4,4′-MDI content of 98 wt. % and a 2,4′-MDI content of 2 wt. % was removed from the divided wall column and discharged as the first fraction. In addition, an overhead stream of 1.2 g/h with a composition of 25 wt. % 2,2′-MDI, 73 wt. % 2,4′-MDI and 2 wt. % 4,4′-MDI was removed together with a side stream of 10.7 g/h with a composition containing 54.9 wt. % 2,4′-MDI and 45.1% 4,4′-MDI.

d) Production of the Second Fraction (Polymeric MDI):

The secondary streams obtained in Example 2 c), i.e. the overhead stream (6 g) and the side stream (51 g) were mixed with the bottom stream (2,050 g) also obtained in Example 2 c) and another batch of di- and polyisocyanates (2,330 g) produced according to Example 2 b). From the mixture thus obtained, 280 g of distillate were again distilled off in a batch distillation at a pressure of 10 mbar and a bottom temperature of 215° C., with 4,157 g of bottom product remaining, having a viscosity of 200 mPas. 2,107 g of this bottom product were removed as a finished product (second fraction) and 2,050 g were returned to be mixed with the overhead and side streams obtained in Example 2 c). The distillate was processed further by distillation in the partition column as in Example 2 c).

In total, with repeated recycling of bottom product and distillate, 223 g of technically pure 4,4′-MDI were obtained as the first fraction and 2,107 g of polymeric MDI with a viscosity of 200 mPas as the second fraction in equilibrium.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the production of 4,4′-diphenylmethane diisocyanate comprising:
a) reacting aniline and formaldehyde in a molar ratio of about 1.7 to about 4:1 in the presence of an acid catalyst to form diphenylmethane series di- and polyamines;
b) reacting the di- and polyamines with phosgene to form corresponding diphenyl methane series di- and polyisocyanates, and optionally separating by distillation, resulting in a mixture of di- and polyisocyanates containing about 44—about 80 wt. % of 4,4′-diphenylmethane diisocyanate together with about 1 to about 12 wt. % of 2,4′- and/or 2,2′-diphenylmethane diisocyanate in total, and about 10 to about 55 wt. % of tri- and polyfunctional polyisocyanates, based on the weight of the mixture of di- and polyisocyanates; and
c) separating the mixture of di- and polyisocyanates by distillation and/or crystallization into precisely two fractions, a first fraction containing about 5—about 40 wt. % of the quantity of the mixture of di- and polyisocyanates and a second fraction containing about 60—about 95 wt. % of the quantity of the mixture of di- and polyisocyanates, and the first fraction containing at least about 97 wt. % of 4,4′-diphenylmethane diisocyanate together with no more than about 3 wt. % of 2,4′-diphenylmethane diisocyanate, based on the weight of the first fraction, and the second fraction containing about 30—about 60 wt. % of 4,4′-diphenylmethane diisocyanate, about 1—about 12 wt. % of 2,4′-diphenylmethane diisocyanate and no more than about 2 wt. % of 2,2′-diphenylmethane diisocyanate together with about 35—about 65 wt. % of tri- and polyfunctional polyisocyanates, based on the weight of the second fraction.

2. The process according to claim 1, in which the distillation and/or crystallization contains several distillation and/or crystallization steps.

3. The process according to claim 1, in which, in step c), a fraction containing at least about 95 wt. % of diisocyanates, based on the weight of the fraction, is initially separated from the mixture of di- and polyisocyanates by distillation and/or by crystallization, which results in a bottom stream containing di- and polyisocyanates.

4. The process according to claim 3, in which the separated fraction containing at least about 95 wt. % of diisocyanates contains at least about 97 wt. % of 4,4′-diphenylmethane diisocyanate together with no more than about 3 wt. % of 2,4′-diphenylmethane diisocyanate, based on the weight of the separated fraction containing at least 95 wt. % of diisocyanates.

5. The process according to claim 3, in which the separated fraction containing at least about 95 wt. % of diisocyanates is at least partly freed from the 2,2′- and 2,4′-isomers and other impurities in additional distillation and/or crystallization steps, with the formation of secondary streams containing 2,2′-MDI, 2,4′-MDI and/or other impurities.

6. The process according to claim 5, in which the bottom stream containing di- and polyisocyanates and the secondary streams containing 2,2′-MDI, 2,4′-MDI and/or other impurities are combined.

7. The process according to claim 5, in which the bottom stream containing di- and polyisocyanates and the secondary streams containing 2,2′-MDI, 2,4′-MDI and/or other impurities are mixed and the mixture is heated and then separated by distillation, the second fraction being obtained as the bottom stream.

8. The process according to claim 5, in which the bottom stream containing di- and polyisocyanates and the secondary streams containing 2,2′-MDI, 2,4′-MDI and/or other impurities are combined and mixed in with the mixture of di- and polyisocyanates obtained in step b) and the resulting mixture is distilled, the second fraction being obtained as the bottom stream.

9. The process according to claim 1, in which the mixture of di- and polyisocyanates contains about 50—about 75 wt. % of 4,4′-diphenylmethane diisocyanate together with about 2—about 10 wt. % of 2,4′- and/or 2,2′-diphenylmethane diisocyanate in total, and about 20 to about 48 wt. % of tri- and polyfunctional polyisocyanates, based on the weight of the mixture of di- and polyisocyanates.

10. The process according to claim 1, in which the mixture of di- and polyisocyanates contains about 55—about 70 wt. % of 4,4′-diphenylmethane diisocyanate together with about 4—about 9 wt. % of 2,4′- and/or 2,2′-diphenylmethane diisocyanate in total, and about 26—about 40 wt. % of tri- and polyfunctional polyisocyanates, based on the weight of the mixture of di- and polyisocyanates,
